(19) United States
(12) Patent Application Publication
(10) Pub. No.: US 2022/0233819 A1
(43) Pub. Date: Jul. 28, 2022

(54) REPOSITIONABLE MEDICAL TUBE WITH ULTRASONICALLY-DETECTABLE CUFF

(71) Applicant: THE REGENTS OF THE UNIVERSITY OF MICHIGAN, Ann Arbor, MI (US)

(72) Inventors: John Charpie, Ann Arbor, MI (US); Adrienne R. Harris, Clinton, MI (US); Shepherd Smith, Milan, MI (US); Kegan McArthur, Saline, MI (US); Gene Parunak, Saline, MI (US)

(21) Appl. No.: 17/617,837

(22) PCT Filed: Jun. 9, 2020
(86) PCT No.: PCT/US20/36732

§ 371 Date: Dec. 9, 2021

Related U.S. Application Data
(60) Provisional application No. 62/859,518, filed on Jun. 10, 2019.

(51) Int. Cl. A61M 25/01; A61M 16/04; A61J 15/00

(52) U.S. Cl. CPC A61M 25/0108; A61M 16/0445; A61J 15/0003; A61M 25/1002; A61M 16/0438; A61M 16/0459; A61J 15/0049

(57) ABSTRACT

A repositionable medical tube, such as an endotracheal tube, a nasogastric tube, or a nasojejunal tube, having an inflatable cuff with at least one divot region therein, the divot region having an outer diameter sufficiently smaller than an outer diameter of both a proximate cuff region and of a distal cuff region as to produce a discernibly-distinct ultrasonic image when scanned by an ultrasound sensor. The medical tube may be further provided with an ultrasonically-detectable coil or one or more ultrasonically-detectable markers having an identifiable geometric shape when the marker is visible using an ultrasonic probe.

### CROSS-REFERENCE TO RELATED APPLICATIONS

This application claims priority to, and the benefit of the filing date of, U.S. Provisional Application No. 62/859,518, filed Jun. 10, 2019, which is hereby incorporated by reference in its entirety.

### STATEMENT OF FEDERALLY SPONSORED RESEARCH AND DEVELOPMENT

This invention was made with government support under Contract No. FD-003787 awarded by the US Food and Drug Administration. The government has certain rights in the invention.

### FIELD OF THE DISCLOSURE

This disclosure relates generally to repositionable medical tubes, such as endotracheal tubes (ETTs) and, more specifically, to medical tubes having one or more inflatable cuffs with geometric features to promote ultrasonic detection and facilitate verification of the tube's location, e.g. in the case of an ETT, tracheal versus esophageal position and depth relative to vocal cords and carina, without need for chest radiography.

### DESCRIPTION OF THE PRIOR ART

Currently, there are more than 25,000 inpatient critical care beds in more than 1,900 neonatal and pediatric intensive care units (ICUs) across the United States. Respiratory illnesses and infections are the most common admitting diagnoses, often requiring endotracheal intubation and mechanical ventilation. Direct laryngoscopy, capnography, and auscultation are routinely used for initial placement of endotracheal tubes (ETTs) in children, and chest radiography (x-ray) is the standard practice to confirm ETT position (trachea vs. esophageal) and depth (relative to the vocal chords and carina). Subsequently, mechanically ventilated neonatal and pediatric ICU patients undergo frequent (sometimes daily) chest x-rays to confirm ETT position due to the serious and potentially life-threatening consequences of unplanned/unrecognized extubations and malpositioned ETTs. The rate of malpositioned ETTs in neonates is as high as 29-50%, and between 0.72 and 4.4 unplanned extubations occur in pediatric patients for every 100 ventilator days.

Incorrect ETT placement can result in vocal cord injury, barotrauma, hypoxia, neurologic injury, and death. Critically ill pediatric patients are at increased risk for these complications due, in part, to their higher oxygen consumption rates than adults. Chest x-rays for the primary purpose of checking ETT position are costly, logistically burdensome, difficult to interpret, and an increased health risk (due to life-time cumulative radiation exposure and late cancers, particularly in children). Among the factors that complicate interpretation of chest x-rays when attempting to verify ETT location, particularly in neonatal and pediatric patients, is the presence of a plurality of lumens, wires, cuffs, bandages, gauze, medical tape, and other paraphernalia that, particularly in a two-dimensional image such as an x-ray, renders the ETT, and components thereof, virtually indistinguishable from one or more of those other objects. These technical challenges associated with chest x-rays could be mitigated by the use of a rapid, readily available, and highly reliable non-radiographic imaging system.

### SUMMARY OF THE DISCLOSURE

A repositionable medical tube of the present disclosure, which may be in the form of an endotracheal tube (ETT), a nasal airway tube such as a nasogastric tube, or a nasojejunal tube, for example, includes at least one inflatable cuff having a geometric topography that facilitates detection by ultrasound to confirm the position of the medical tube. An ultrasonic transducer can be operated to transmit signals intended for use in detecting the presence (or absence) of foreign objects at specific depths under the skin of a patient. This ability to focus ultrasonic energy to a specific depth provides an opportunity to discern specific objects within the patient while mitigating the risk of confusion of the medical tube with other lumens at different depths under the skin of the patient.

In an exemplary embodiment, the cuff of a medical tube, such as an ETT, is provided with a circumferential, preferably toroidal, divot region, wherein the outer diameter of the cuff of the medical tube is reduced from a first diameter (which is of a diameter to engage an inner diameter of the trachea when inflated, preferably with saline) to a second diameter that is sufficiently less than the first diameter to provide, when the medical tube is placed in the trachea of a patient, a perceptible ultrasonic signature distinct from an ultrasonic signature of regions of the cuff of the medical tube distal and proximal of the divot region is generated. The axial extent of the divot region also must be of sufficient length to detect an ultrasonic signature distinct from the adjacent proximate and distal regions of the cuff.

In certain embodiments, the medical tube, such as an ETT, may be provided with one or more ultrasonically-detectable coils in the region of the inflatable cuff. Each of the coil(s), which may be metal or another radiopaque or echogenic material, is visible in the region of the inflatable cuff only when the cuff is filled with a liquid, such as water or saline. In embodiments where the inflatable cuff is divoted and that also are provided with one or more ultrasonically-detectable coils, the coil(s) serve as a redundant indicator. In other embodiments in which no divot is provided in the inflatable cuff, the coil serves as the primary indicator of ETT position. The coil(s) may be provided on an exterior of the primary lumen of the medical tube about which the inflatable cuff extends. Alternatively, the coil(s) may be embedded within the wall of the primary lumen about which the inflatable cuff extends.

In certain additional embodiments, the ultrasonically-detectable marker may not be a coil at all, but rather, may be an ultrasonically-detectable indicator in any suitable shape. Such an indicator may be provided on one or both sides of a divot within an inflatable cuff of a divoted cuff, may extend across a region coinciding with the location of the divot, or may be provided within a non-divoted inflatable cuff. As is the case with the ultrasonically-detectable coil(s) described above, the alternately-shaped ultrasonically-detectable marker(s) may be provided on an exterior of the primary lumen of the medical tube about which the inflatable cuff extends, or alternatively, the alternately-shaped ultrasonically-detectable marker(s) may be embedded within the wall of the primary lumen about which the inflatable cuff extends.

### BRIEF DESCRIPTION OF THE SEVERAL VIEWS OF THE DRAWINGS

FIG. 1 is a cross-sectional view, taken along lines 1-1 of FIG. 2, of a cuff portion of a repositionable medical tube of a first embodiment of the present disclosure, the cuff portion having a toroidal divot region;

FIG. 2 is a side view of a cuff portion of a repositionable medical tube of the first embodiment of the present disclosure illustrating the cuff portion in an inflated condition, the cuff portion having a toroidal divot region;

FIG. 3 is a screenshot of an ultrasound image of a repositionable medical tube of the type illustrated in FIG. 2, at a position along the medical tube distally of a distal end of the cuff portion;

FIG. 4 is a screenshot of an ultrasound image of the repositionable medical tube of the type illustrated in FIG. 2, at a position along the cuff portion that is along a maximum diameter region of the cuff distally of the toroidal divot region and proximate to the distal end of the cuff portion;

FIG. 5 is a screenshot of an ultrasound image of the repositionable medical tube of the type illustrated in FIG. 2, at a position along the cuff portion that is along the toroidal divot region of the cuff portion;

FIG. 6 is a screenshot of an ultrasound image of the repositionable medical tube of the type illustrated in FIG. 2, at a position along the cuff portion that is along a maximum diameter region of the cuff proximate to the toroidal divot region and distal of a proximate and of the cuff portion;

FIG. 7 is a screenshot of an ultrasound image of the repositionable medical tube of the type illustrated in FIG. 2, at a position along the medical tube proximate to the proximate and of the cuff portion;

FIG. 8 is a cross-sectional view, taken along lines 8-8 of FIG. 9, of a cuff portion of a repositionable medical tube of a second embodiment of the present disclosure, wherein the divot is asymmetrical relative to the cuff, i.e. the divot is located at a longitudinal position other than a midpoint of the cuff;

FIG. 9 is a side view of the cuff portion of the repositionable medical tube of the second embodiment of the present disclosure;

FIG. 10 is a cross-sectional view, taken along lines 10-10 of FIG. 11, of a cuff portion of a repositionable medical tube of a third embodiment of the present disclosure, wherein the cuff includes two divot portions;

FIG. 11 is a side view of the cuff portion of the repositionable medical tube of the third embodiment of the present disclosure;

FIG. 12 is a cross-sectional view, taken along lines 12-12 of FIG. 13, of a repositionable medical tube of a fourth embodiment of the present disclosure, which includes two independent cuff regions axially spaced from one another, each of the cuff regions having a respective inflation lumen;

FIG. 13 is a side view of the repositionable medical tube of the fourth embodiment of the present disclosure, including two independent cuff regions;

FIG. 14 is a cross-sectional view, taken along lines 14-14 of FIG. 15, of a cuff portion of a repositionable medical tube of a fifth embodiment of the present disclosure, which includes two independent cuff regions that are axially substantially co-extensive with one another, but radially spaced from one another, each of the cuff regions having a respective inflation lumen, resulting in a pair of non-toroidal, axially-extending divots separating the independent cuff regions;

FIG. 15 is a side view of the cuff region of the repositionable medical tube of the fifth embodiment of the present disclosure;

FIG. 16 is an end view of the cuff region of the fifth embodiment of the medical tube of the present disclosure;

FIG. 17 is a cross-sectional view of a sixth embodiment of a medical tube of the present disclosure, wherein a pair of ultrasonically-detectable (e.g., metal) coils are disposed within a centrally-devoted inflatable cuff region of the medical tube, one on either side of the divot;

FIG. 18 is a side view of the medical tube of the sixth embodiment, with a portion of the centrally-divoted inflatable cuff removed to illustrate the ultrasonically-detectable coils therein;

FIG. 19 is a cross-sectional view of a seventh embodiment of a medical tube of the present disclosure, wherein an ultrasonically-detectable (e.g., metal) coil is disposed within a centrally-divoted inflatable cuff region of the medical tube, the ultrasonically-detectable coil running substantially the length of the inflatable cuff, including a region underlying the divot;

FIG. 20 is a side view of the medical tube of the seventh embodiment, with a portion of the centrally-divoted inflatable cuff removed to illustrate the ultrasonically-detectable coil therein;

FIG. 21 is a cross-sectional view of an eighth embodiment of a medical tube of the present disclosure, wherein a single ultrasonically-detectable (e.g., metal) coil is disposed within a non-divoted inflatable cuff region of the medical tube, the coil extending substantially the length of the inflatable cuff;

FIG. 22 is a side view of the medical tube of the eighth embodiment, with a portion of the centrally-divoted inflatable cuff removed to illustrate the ultrasonically-detectable coil therein;

FIG. 23 is a side view of a ninth embodiment of a medical tube of the present disclosure, with a portion of a centrally-divoted inflatable cuff removed to illustrate a non-coil ultrasonically-detectable marker therein;

FIG. 24 is a cross-sectional view of the ninth embodiment of the medical tube of the present disclosure;

FIG. 25 is a side view of a tenth embodiment of a medical tube of the present disclosure, with a portion of a non-divoted inflatable cuff removed to illustrate a non-coil ultrasonically-detectable marker therein;

FIG. 26 is a cross-sectional view of the tenth embodiment of the medical tube;

FIG. 27 is an image of an ultrasound probe on a model of anatomic tissue with a medical tube of the present disclosure provided with an ultrasonically-detectable coil;

FIG. 28 is an ultrasonic image of a medical tube made in accordance with the eighth embodiment of the present disclosure, wherein the inflatable cuff is filled with air, such that a portion of the ultrasonically-detectable coil that is not underlying the inflatable cuff is visible and a portion of the ultrasonically-detectable coil that is underlying the inflatable cuff is not visible; and

FIG. 29 is an ultrasonic image of a medical tube made in accordance with the eighth embodiment of the present disclosure, wherein the inflatable cuff is filled with water, such that the ultrasonically-detectable coil is visible inside the inflatable cuff.

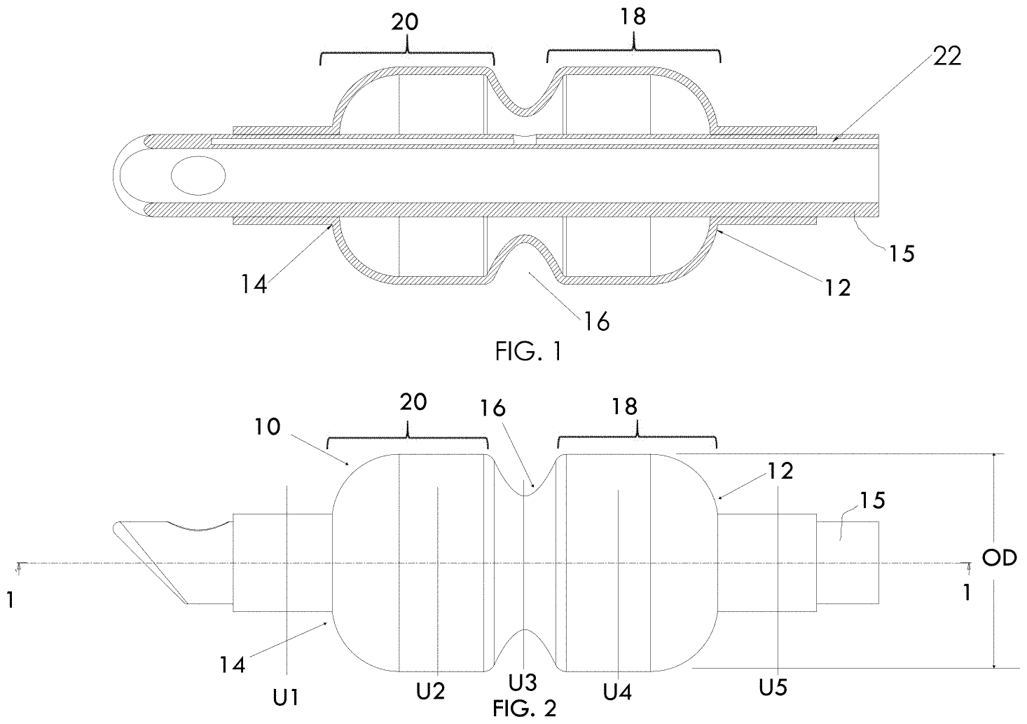

### DETAIL DESCRIPTION OF THE PREFERRED EMBODIMENTS

In a preferred embodiment of the present disclosure, a repositionable medical tube, such as an endotracheal tube (ETT), as illustrated in FIG. 2, is provided with a fluid-filled cuff 10. The cuff 10 has a proximate end 12 and a distal end 14. The cuff 10 is provided with a toroidal divot region 16 intermediate the proximate end 12 and the distal end 14, having an outer diameter smaller than outer diameters of a proximate cuff region 18 intermediate the proximate end 12 and the toroidal divot region 16, and a distal cuff region 20 intermediate the toroidal divot region 16 and the distal end 14. The outer diameter of the toroidal divot region 16 is sufficiently smaller than the diameters of the proximate cuff region 18 and distal cuff region 20, and the axial extent of the toroidal divot region 16 is of sufficient length, such that when the ETT is placed in the trachea of a patient, a perceptible ultrasonic signature distinct from an ultrasonic signature of regions of the cuff of the ETT distal and proximal of the divot region is generated. Providing the cuff 10 with a single toroidal divot region 16 gives the cuff 10 the general appearance of a dog bone. However, as discussed in greater detail below with respect to alternate embodiments of the disclosure, it will be appreciated that multiple divot regions could be provided at desired axial locations along the cuff, or the divot could be asymmetric, i.e. off-centered relative to the proximate cuff region 18 and the distal cuff region 20, which can improve precision in locating features above and below the cuff 10.

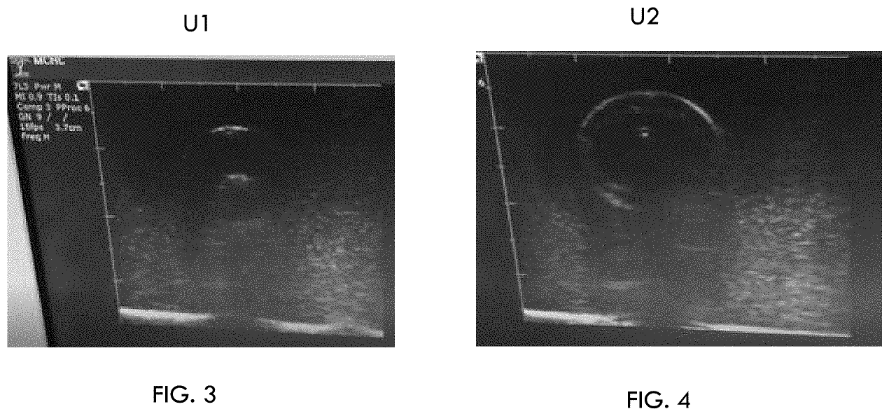

Turning now to FIGS. 3-7, a series of ultrasound images are provided that collectively illustrate the manner in which the provision of a fluid-filled cuff 10 (which fluid may be a gas, such as air, or a liquid, such as saline) having a toroidal divot region 16 facilitates accurate confirmation of ETT location in situ. The fluid-filled cuff 10 of the present disclosure leverages the principal of acoustical mismatch to create an identifiable marker. The acoustic impedance of soft human tissue is 1.3-1.6. The acoustic impedance of bone is 7.8. The acoustic impedance of air is 0.0004, and that of saline is approximately 1.5 (10⁶*Kg/m²*s). While the acoustical mismatch between the air-filled divot and the fluid-filled cuff 10 may provide sufficient differentiation to identify the divot, the wall material of the fluid-filled cuff 10 may additionally be made of an ultrasonically-reflective material to enhance the acoustical mismatch, facilitating the identification of the location of the divot region 16. An ultrasound probe is tunable to a particular depth. This capability advantageously provides an ability to reliably avoid noise or extraneous signal interference from the presence of various other tubes and leads that may be positioned on, or implanted in, a patient that might cause confusion when attempting to accurately discern ETT location from reading conventional x-ray images.

Where the acoustic impedance of a marker is higher than the surrounding tissue, a positive trace or signal response is generated, and negative where the acoustical impedance of a marker is lower than the surrounding tissue.

FIG. 3 is an ultrasound image generated using an ultrasound probe, such as an ACUSON 7L3 4.5/5.4/6.6 MHz Linear Transducer for Cypress, manufactured by Siemens (headquartered in Malvern, Pa., with its Ultrasound Division in Mountain View, Calif.) tuned to a depth of 2-3 cm, with variable gain, detecting the ETT at a location U1, just distally of the distal end 14. The frequency of the ultrasound probe that is used and the ultrasound machine settings, such as gating and tuning depth, would be optimized for image quality primarily based on the size of the patient. No outline of the cuff 10, which is filled with saline, can be discerned from the ultrasound image of FIG. 3. FIG. 4 is an ultrasound image of a location U2, along the distal cuff region 20. The cuff outline is discernible in the ultrasound image of FIG. 4.

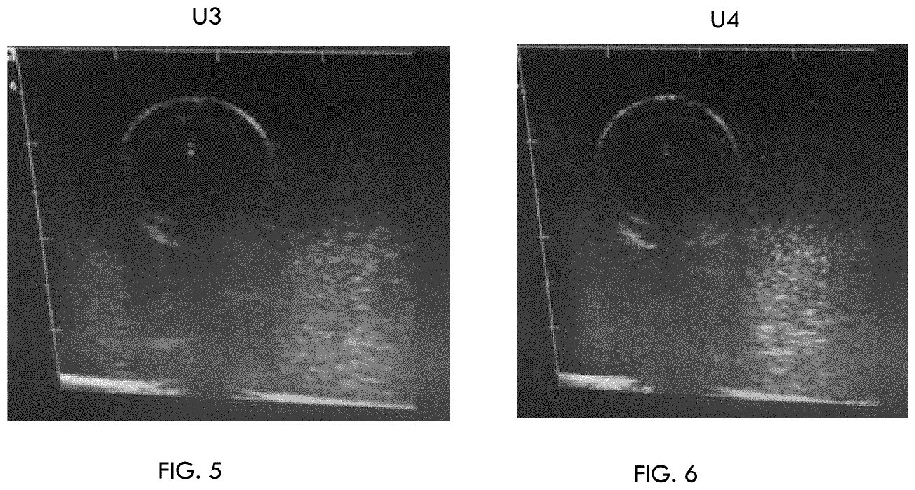

FIG. 5 is an ultrasound image of a location U3, along the toroidal divot region 16 of the cuff 10. The outline of the cuff 10 is not discernible in the ultrasound image of FIG. 5.

FIG. 6 is an ultrasound image of a location U4, along the proximate cuff region 18. As in FIG. 4, the outline of the cuff 10 is discernible in the ultrasound image of FIG. 6.

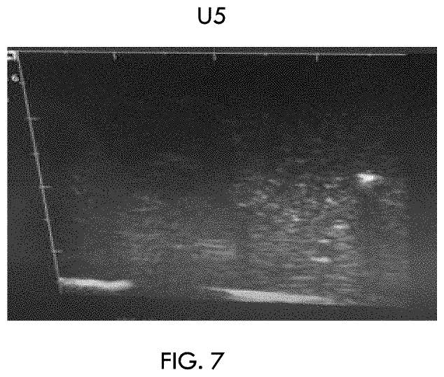

FIG. 7 is an ultrasound image of a location U5, just proximately of the proximate end 12. As in FIG. 3, the outline of the cuff 10 is not discernible in the ultrasound image of FIG. 7.

Once a clinician, operating an ultrasound sensor set to the appropriate depth, detects a transition, i.e. a change indicative of the ultrasound sensor having moved from a location in which the presence of the cuff is not detected to a location in which the presence of the cuff 10 is detected, the clinician can continue moving the ultrasound sensor a distance commensurate with about ½ the axial length of the cuff 10. Upon seeing a further change indicative of the ultrasound sensor having moved from a location where the presence of a portion of the cuff 10 is detected to a location where the presence of the cuff 10 is no longer detected, then a further change indicative of the ultrasound sensor having moved to a location where the presence of a portion of the cuff 10 is again detected as the clinician continues to move the ultrasound sensor in the same direction, the clinician can interpret the dynamic changes in the ultrasound image. Specifically, the location of the ultrasound sensor coinciding with the absence of a discernible signal indicative of the presence of the cuff 10 between portions superiorly and inferiorly that coincide with the presence of a discernible signal indicative of the presence of the cuff 10, is indicative of the exact location of the toroidal divot region 16. This informs the clinician of the exact location of the cuff 10, and thus the location of the ETT, without the need to reposition the patient for purposes of confirming ETT location via radiography. Unnecessary exposure to radiation is also avoided.

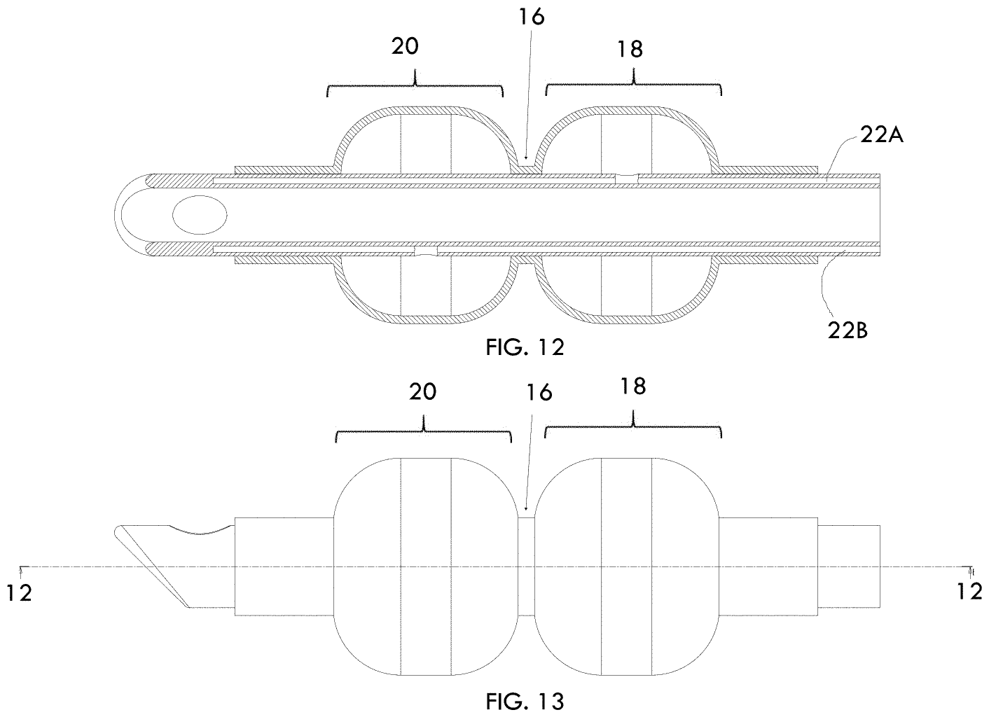

A divot region 16 having an axial length of 5 mm is preferred, and provides approximately 2.5 mm resolution. The cuff 10 is preferably inflated to a standard pressure of 25 cm H20. The toroidal divot region is substantially devoid of inflation medium, such as air or saline. The cuff 10 may be provided with one or more inflation lumens to introduce an inflation medium to an interior of the cuff 10 (so as to inflate) or withdraw inflation medium from the interior of the cuff 10 (to deflate). The proximate cuff region 18 and distal cuff region 20, as well as the divot region 16, may be in fluid communication with one another and with a single inflation lumen 22. Alternately, such as discussed below with respect to an alternate embodiment illustrated in FIGS. 12 and 13, independent inflation lumens may be associated with the proximal cuff region 18 and distal cuff region 20, respectively, with at least a narrowest section of the divot region defined by a lumen wall adhered directly to an exterior of a primary lumen 15 of the medical tube. The inflation lumen 22 may be substantially coextensive with, and preferably incorporated into, a wall of the primary lumen 15 of the medical tube. The cuff 10 may be sufficiently oblong so as to facilitate repositioning of the ETT without the need to deflate the cuff 10. Because of the improved precision in determining the location of the cuff 10 afforded by the toroidal divot region 16, a clinician can reposition the cuff 10 to a desired location without deflation, if desired.

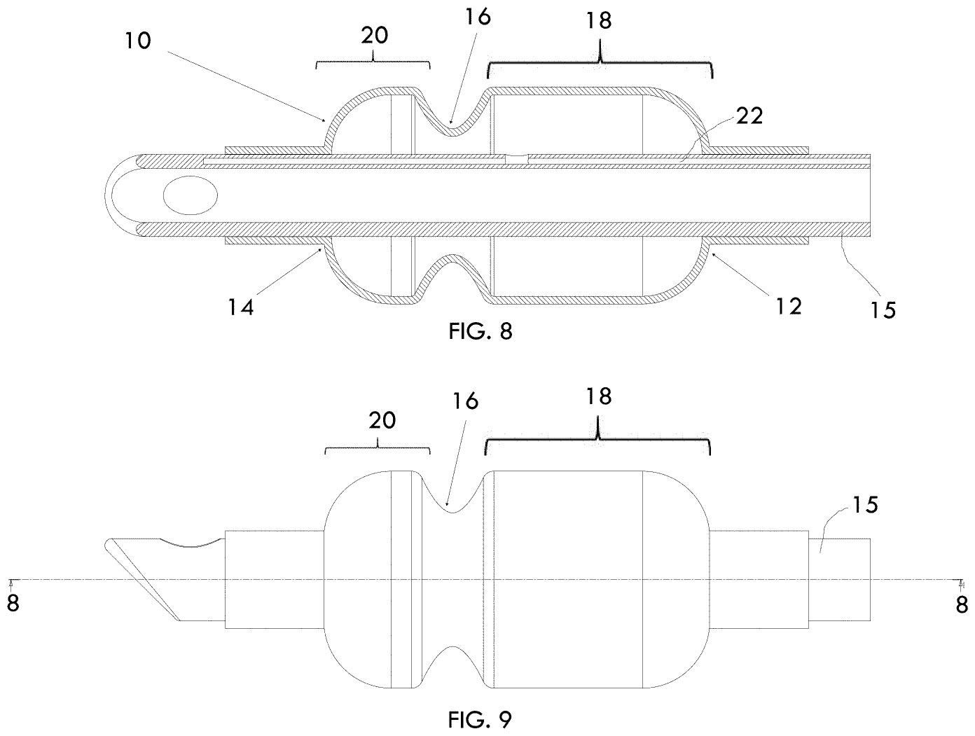

The location of the divot along the length of the cuff 10 need not be centered. For instance, the divot might instead be located ⅔ of the distance from the proximal-to-distal end, or ⅓ of the distance from the proximal-to-distal end of the cuff 10. A second embodiment of a repositionable medical tube of the present disclosure is illustrated in FIGS. 8 and 9. According to this embodiment, the divot region 16 is asymmetric relative to the proximate cuff region 18 and distal cuff region 20. This is accomplished by providing a relatively longer proximate cuff region 18 (in the axial direction) and a relatively shorter distal cuff region 20. As in the case of the first embodiment, the proximate cuff region 18, the distal cuff region 20, and the divot region 16 are in fluid communication with one another and share a common single inflation lumen 22.

A benefit of having the divot region 16 located closer to the distal end 14 of the cuff 10 than the proximal end 12 is that the location facilitates the clinician confirming the location of the divot more quickly, as the ultrasound sensor is moved from a location distally of the distal end 14 toward the proximal end 12 of the cuff 10. Another benefit is that, without increasing the overall length of the cuff 10, the clinician can see the ultrasonic signature of the longer proximate cuff region 18 as distinguished from the signature of the relatively short distal cuff region 20 to better assess the orientation of the cuff 10 and verify whether the extent of migration, if any, of the medical tube is within acceptable parameters, all without the need of repositioning the patient for x-rays.

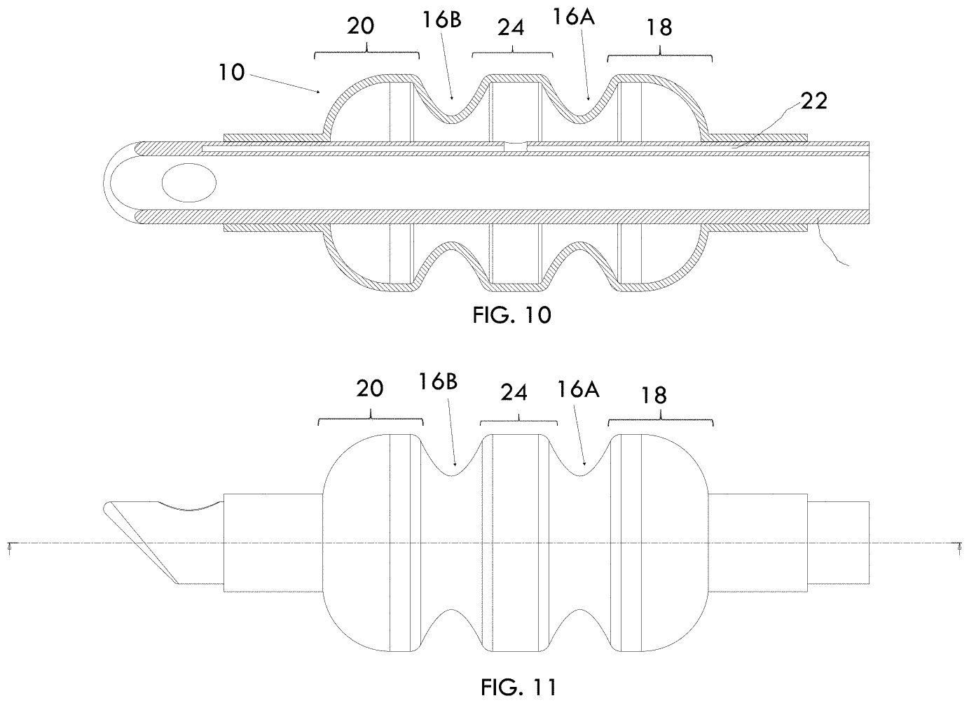

While the cuff has been illustrated as having a single divot, an alternate embodiment is for the cuff to be provided with multiple divots, such as illustrated in FIGS. 10 and 11. According to this third embodiment, the cuff 10 includes a proximate cuff region 18, a distal cuff region 20, a central cuff region 24, a first, proximal divot region 16A, disposed between the central cuff region 24 and the proximate cuff region 18, and a second, distal divot region 16B, disposed between the central cuff region 24 and the distal cuff region 20. The proximate cuff region 18, the distal cuff region 20, the central cuff region 24, the first, proximal divot region 16A, and the second, distal divot region 16B are in fluid communication with one another and share a common single inflation lumen 22.

A fourth embodiment is illustrated in FIGS. 12 and 13. According to this embodiment, the proximate cuff region 18 and the distal cuff region 20 are not in fluid communication with one another. Instead, the proximate cuff region 18 may be selectively inflated and deflated via a proximate cuff inflation lumen 22A with which the proximate cuff region 18 is in fluid communication, and the distal cuff region 20 may be selectively inflated and deflated via a distal cuff inflation lumen 22B with which the distal cuff region 20 is in fluid communication. The distal cuff inflation lumen 22B is fluidly isolated from the proximate cuff inflation lumen 22A. Both the proximate cuff inflation lumen 22A and the distal cuff inflation lumen 22B may be substantially coextensive with the primary lumen 15 of the medical tube, and preferably incorporated into the wall of the primary lumen 15. According to this embodiment, the divot region 16 is defined between the proximate cuff region 18 and the distal cuff region 20, but is not inflatable. Rather, an outer wall of the distal cuff region is formed directly outboard of the outer wall of the primary lumen 15.

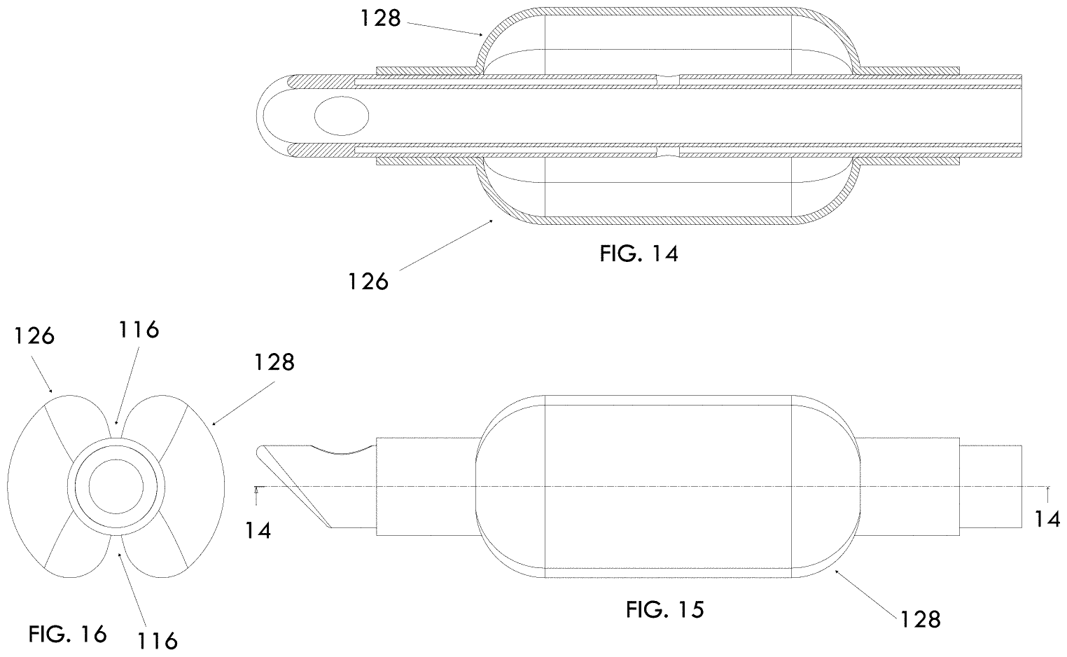

A fifth embodiment of the present disclosure is illustrated in FIGS. 14-16. According to this embodiment, a pair of axially extending divot regions 116 are provided, each of which is axially coextensive with first and second cuff regions 126, 128. A benefit of such one or more axially extending divot regions 116 is that the divot region 116 can be used in concert with an ultrasound probe to detect rotation of the medical tube, which can be useful in guiding or verifying proper advancement as between the trachea versus the esophagus. Rotation of an ultrasound probe through an angle of, for example, about 90° can permit depth readings with the ultrasound probe that would not be achievable using a medical tube lacking such axially-extending divot regions 116. While the first and second cuff regions 126, 128 could both be inflated with the same fluid, it may be desirable to fill the first cuff region 126 with a different fluid than the second cuff region 128, such as filling one of the cuff regions with a liquid (e.g., saline) and the other with a gas, or filling one of the cuff regions with a radiopaque fluid and the other with a radio-transparent fluid.

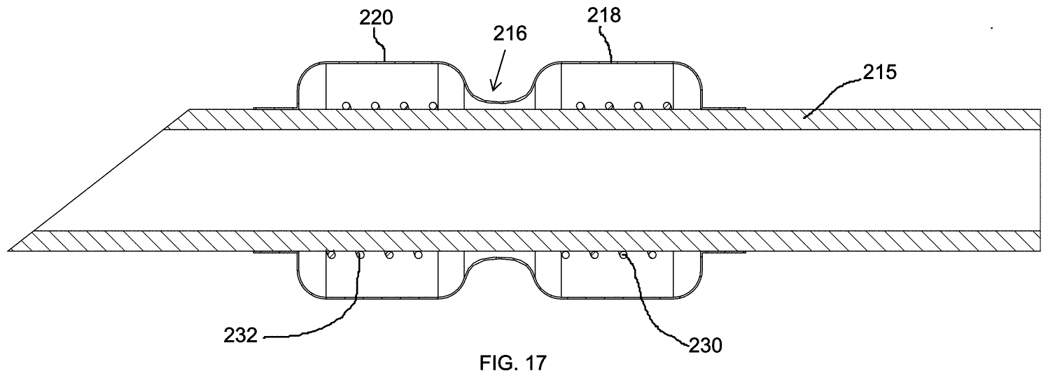

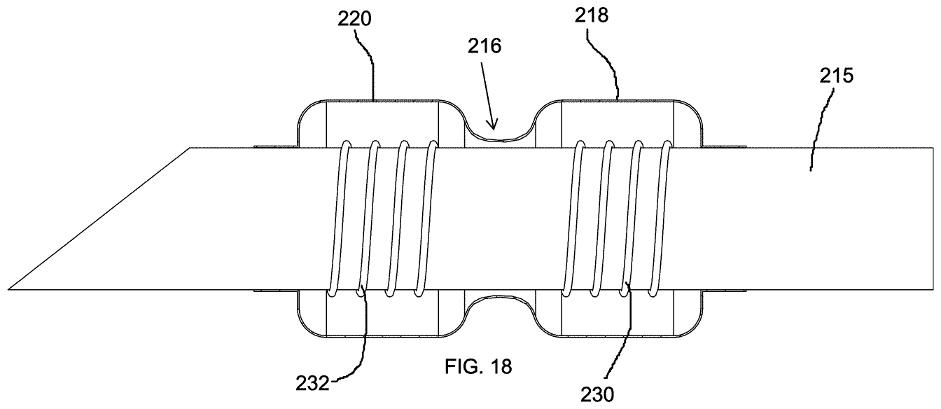

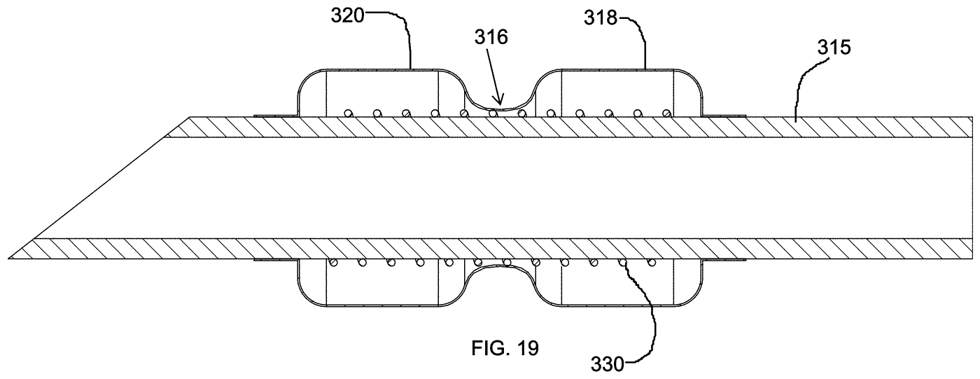

A sixth embodiment of the present disclosure is illustrated in FIGS. 17 and 18. In this embodiment, a medical tube has a primary lumen 215, surrounded by inflatable cuff including a divot region 216, and proximate and distal cuff regions 218, 220. A pair of ultrasonically-detectable coils 230, 232, which are also referred to herein as coil segments, may be made of metal or other suitable material, such as a radiopaque or echogenic material, are disposed about the primary lumen 215. Alternately, the entirety or some portion (such as one or both ends, and/or a middle region) of the coils 230, 232 may be embedded within the primary lumen 215. One of the coils 230 is disposed within the proximate cuff region 218 and the other coil 230 is disposed within the distal cuff region. When the inflatable cuff is filled with water, saline or any other fluid that does not attenuate ultrasound, the coils 230, 232 are ultrasonically detectable, and the ability to discern the gap between the coils 230, 232, which gap coincides with the location of the divot region 216, facilitates identification of the particular location of the inflatable cuff.

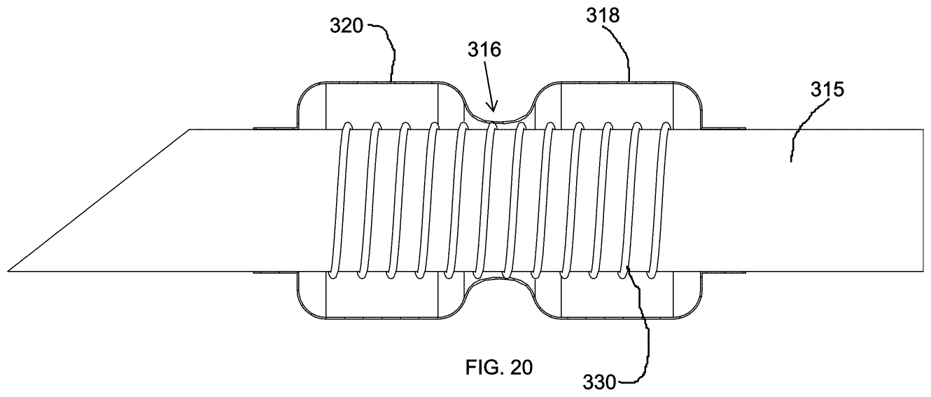

A seventh embodiment is illustrated in FIGS. 19 and 20. This embodiment differs from the sixth embodiment in that there is a single ultrasonically-detectable coil 330 extending substantially the length of the inflatable cuff. The coil 330 is disposed about the primary lumen 315 and extends not only under each of the proximal and distal inflatable cuff regions 318, 320, but also under the divot region 316. Alternately, the entirety or some portion (such as one or both ends, and/or a middle region) of the coil 330 may be embedded within the primary lumen 315. An advantage of this embodiment is its relative ease of manufacture, in that only a single, continuous coil component needs to be secured to (or within) the primary lumen, as opposed to multiple coil segments. When the proximate and distal cuff regions 318, 320 are evacuated of air, or filled with water, saline, or any other fluid that does not attenuate ultrasound, the entire length of the coil 330 is detectable via ultrasound.

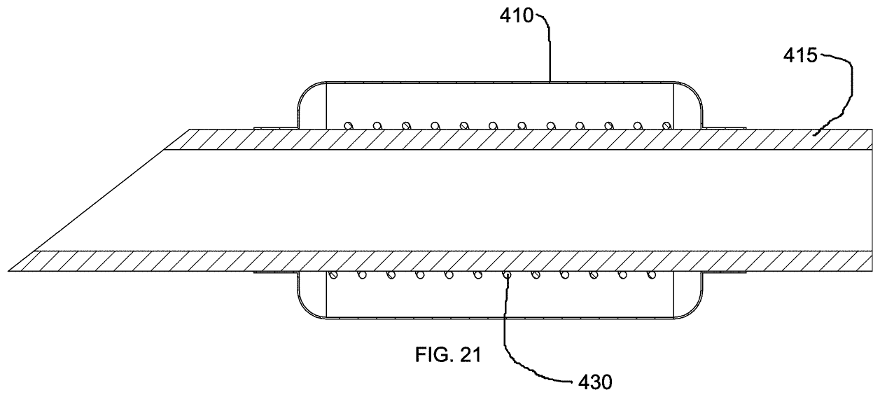

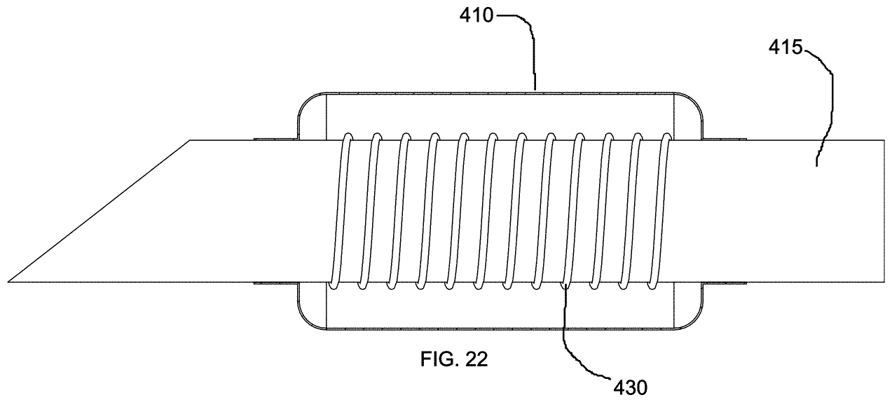

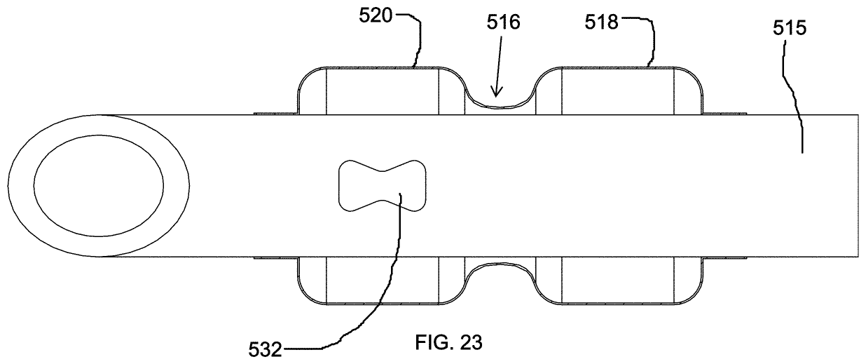

An eighth embodiment is illustrated in FIGS. 21 and 22. According to this embodiment, a non-divoted inflatable cuff 410 is provided and a single ultrasonically-detectable coil 430 is disposed about a primary lumen 415, the coil 430 preferably extending substantially the length of the inflatable cuff 410. Alternately, the entirety or some portion (such as one or both ends, and/or a middle region) of the coil 430 may be embedded within the primary lumen 415.

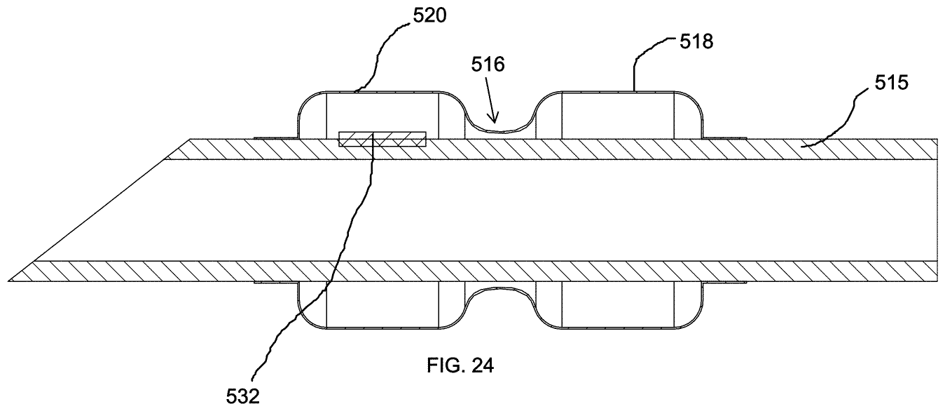

According to a ninth embodiment, illustrated in FIGS. 23 and 24, a tube with a primary lumen 515 and an inflatable cuff having a proximate region 518, a distal region 250, and a divot 516, which is preferably a centrally-located divot, is provided. According to this embodiment, the ultrasonically-detectable element is not a coil, but rather, is a marker 532 that is constructed of ultrasonically-detectable material, such as metal or other suitable material, such as a radiopaque or echogenic material, of a suitable size and shape as to aid in ultrasonically discerning the position of the inflatable cuff in vivo. While the shape of the marker 532 may vary, the shape is preferably a shape that is readily distinct from shapes found in regions of the human anatomy near where the tube is to be used, so as to avoid confusion. For instance, the marker 532 may be provided with a mesh pattern, may be a plurality of wavy lines, may be a cross-hatched region, or may include other readily identifiable geometric shapes or patterns. In this particular embodiment, the marker 532 is positioned on, and/or embedded in, the primary lumen 515 within the distal region 520 of the inflatable cuff. The ultrasonic signature of the divot region and of the marker 532, when the inflatable cuff is filled with water, saline, or any other fluid that does not attenuate ultrasound, can be used provide ultrasonic verification of position of the inflatable cuff. Alternatively, a plurality of markers may be provided, with a first marker 532 placed in one portion of the divot and a second marker 532 placed in another. The first and second markers may be of the same geometric shape or different shapes. A benefit of the markers being of different shapes is that the shapes of the markers can serve as an indicator to the clinician as to the orientation of the tube.

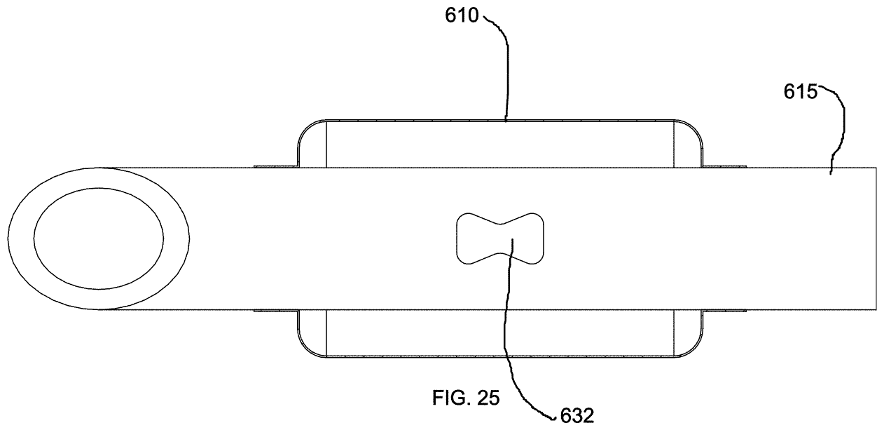

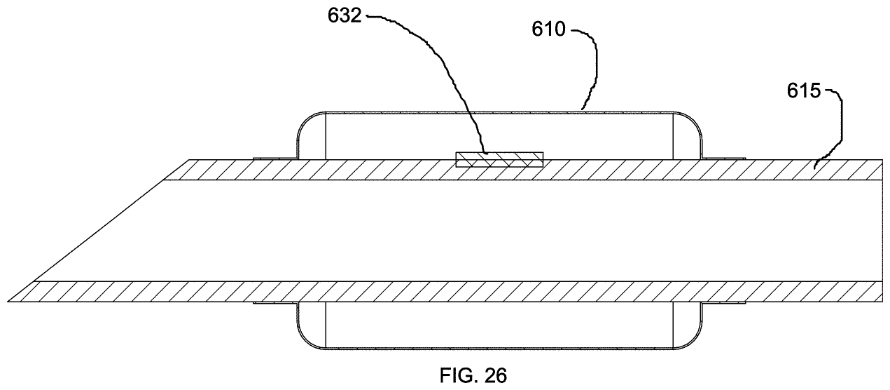

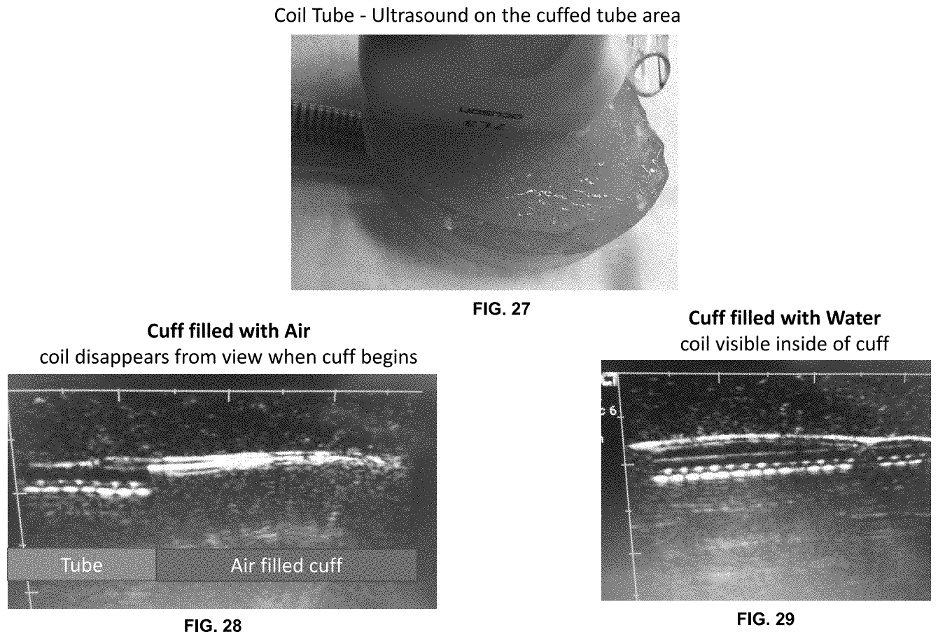

In a tenth embodiment, illustrated in FIGS. 25 and 26, a non-divoted inflatable cuff 610 is provided. A marker 632 is provided on (and/or embedded in) a primary lumen 615. In this embodiment, the marker 632 is centrally located relative to an axial extent of the inflatable cuff 610, such that the ultrasonic signature of the marker 632 indicates that half of the inflatable cuff 610 is distal of the marker's location indicated by the signature, and half of the inflatable cuff is proximate of location of the marker's location indicated by the signature. If desired, a plurality of markers 632 could be provided along the length of the inflatable cuff. The markers 632 may be of the same geometric shape or different shapes, with the different shapes serving as an indicator to the clinician as to the orientation of the tube. For instance, markers 632 may be provided at locations along the distal, central, and proximate portions of the cuff 610.

While certain embodiments have been described herein, it will be appreciated that variations can be made thereto that are still considered within the scope of the appended claims.

What is claimed is:

1. A medical tube having an inflatable cuff, the inflatable cuff comprising:
a proximate end;
a proximate cuff portion adjacent the proximate end;
at least one divot portion disposed distally of the proximal cuff portion;
a distal cuff portion disposed opposite the at least one divot portion from the proximate cuff portion; and
a distal end adjacent the distal cuff portion.

2. The medical tube of claim 1, wherein in the inflatable cuff, the proximal cuff portion has a first maximum outer diameter, the distal cuff portion has a second maximum outer diameter, and the divot portion has third outer diameter is sufficiently less than the first and second outer diameters to produce, when scanned using an ultrasound sensor, a discernibly-distinct ultrasonic image from ultrasonic images of the proximate cuff portion and the distal cuff portion.

3. The medical tube of claim 2, wherein in an ultrasonic image produced by an ultrasound sensor positioned over at least one of the proximate cuff portion and the distal cuff portion, a signal indicative of the presence of the inflatable cuff is discernible.

4. The medical tube of claim 2, wherein in the discernibly-distinct ultrasonic image produced by an ultrasound sensor positioned over the at least one divot portion, a signal indicative of the presence of the inflatable cuff is not discernible.

5. The medical tube of claim 1, further including an inflation lumen in fluid communication with an interior of the inflatable cuff.

6. The medical tube of claim 5, the inflatable cuff filled with air.

7. The medical tube of claim 1, the inflatable cuff filled with saline.

8. The medical tube of claim 1, wherein the at least one divot portion is generally toroidal.

9. The medical tube of claim 1, wherein the medical tube is an endotracheal tube.

10. The medical tube of claim 1, wherein the medical tube is a nasogastric tube.

11. The medical tube of claim 1, wherein the medical tube is a nasojejunal tube.

12. The medical tube of claim 1, further comprising a first inflation lumen in fluid communication with the proximate cuff portion and a second inflation lumen in fluid communication with the distal cuff portion.

13. The medical tube of claim 1, wherein the proximate cuff region is of a first axial length and the distal cuff region is of a second axial length that is less than the first axial length, whereby the divot region is disposed closer to the distal end of the medical tube than to the proximate end of the medical tube.

14. The medical tube of claim 1, further comprising a central cuff region separating the at least one divot region into a first, proximate divot region between the central cuff region and the proximate cuff region, and a second, distal divot region between the central cuff region and the distal cuff region.

15. The medical tube of claim 1, further comprising an ultrasonically-detectable coil within the inflatable cuff, the coil extending along a primary lumen about which the inflatable cuff is disposed.

16. The medical tube of claim 15, wherein the coil extends substantially the length of the inflatable cuff.

17. The medical tube of claim 15, wherein the ultrasonically-detectable coil includes a first ultrasonically-detectable coil segment extending substantially the length of the proximate cuff portion and a second ultrasonically-detectable coil segment extending substantially the length of the distal cuff portion, with no coil segment underlying the divot portion of the inflatable cuff.

18. The medical tube of claim 1, further comprising an ultrasonically-detectable marker provided on a primary lumen about which the inflatable cuff is disposed, said marker having a geometric shape that is identifiable under conditions in which the marker is visible via an ultrasound probe.

19. The medical tube of claim 18, the marker disposed underlying one of the proximate cuff portion, the distal cuff portion, or the divot region of the inflatable cuff.

20. A medical tube having a first inflatable cuff and a second inflatable cuff, the first and second inflatable cuffs separated from one another by at least one axially-extending divot region extending an entire length of at least one of the first and the second inflatable cuffs.

21. The medical tube of claim 20, further comprising an ultrasonically-detectable coil segment within each of the first and second inflatable cuffs, the coil segments extending along a primary lumen about which the first and second inflatable cuffs are disposed.

22. The medical tube of claim 21, wherein one of the coil segments extends substantially the length of each of the inflatable cuffs.

23. The medical tube of claim 21, further comprising an additional coil segment within the divot region extend between and connecting to the coil segments within the first and second inflatable cuffs.

24. The medical tube of claim 20, further comprising an ultrasonically-detectable marker provided on a primary lumen about which the inflatable cuffs are disposed, said marker having a geometric shape that is identifiable under conditions in which the marker is visible via an ultrasound probe.

25. The medical tube of claim 24, the marker disposed underlying one of the proximate first inflatable cuff or the second inflatable cuff.
